(19) United States
(12) Patent Application Publication
(10) Pub. No.: US 2017/0347868 A1
(43) Pub. Date: Dec. 7, 2017

(54) MONITORING AND TREATMENT OF GASTROINTESTINAL CONDITIONS

(71) Applicants: Alexander Martin ZOELLNER, Menlo Park, CA (US); Michael D. LAUFER, Menlo Park, CA (US)

(72) Inventors: Alexander Martin ZOELLNER, Menlo Park, CA (US); Michael D. LAUFER, Menlo Park, CA (US)

(21) Appl. No.: 15/175,442

(22) Filed: Jun. 7, 2016

(51) Int. Cl. A61B 1/04; A61B 1/00; A61B 1/32; A61M 13/00

(52) U.S. Cl. CPC A61B 1/041; A61M 13/003; A61B 1/00149; A61B 1/32

(57) ABSTRACT

A tissue treatment system for diagnosing and/or treating gastrointestinal conditions is described where an endoscopic capsule sized for passage through an intestinal tract is used with a jaw assembly. The jaw assembly defines a region for maintaining a position of the endoscopic capsule and the endoscopic capsule comprises at least one actuator which can be activated for enhancing visualization within the intestinal tract.

### FIELD OF THE INVENTION

The field of the subject disclosure relates to gastrointestinal (GI) diagnostics and treatment, more specifically to a new and useful system and method to obtain the location of a GI bleed.

### BACKGROUND OF THE INVENTION

Obscure gastrointestinal bleeding (OGIB) is a bleeding in the gastrointestinal tract at a location that cannot be identified using upper and lower endoscopy. OGIBs are almost always recurrent, and may be caused by lesions in the small intestine. OGIBs are difficult or impossible to detect using endoscopy. This is because an endoscope may be inserted through the rectum, and may not travel far enough into the small intestine to detect any bleeding there. Similarly, endoscope may be inserted through a patient's mouth, and may not be able to inter through the stomach into the small intestine to detect any bleeding there.

In some cases, existence of OGIB may be identified using capsule endoscopy. In such technique, an endoscopic capsule is consumed by a patient, and the endoscopic capsule is transported through the patient's gastrointestinal tract by the patient's body. While the endoscopic capsule travels through the small intestine, it captures images there and transmits the images for recording at an external device. After the endoscopic capsule has exited the body, the recorded images at the external device are then retrieved and analyzed to determine whether there is any bleeding in the gastrointestinal tract. While such technique may allow a physician to determine an existence of an OGIB, it does not allow the physician to determine the location of the OGIB. This is because once the physician identifies an image from the endoscopic capsule that shows a bleeding, the image does not tell the physician the position of the bleeding site.

### SUMMARY OF THE INVENTION

A medical tool for use with an endoscopic capsule includes: a jaw assembly configured to maintain the endoscopic capsule at a certain position or within a range of positions relative to the jaw assembly while the endoscopic capsule is inside a bodily lumen, the jaw assembly having a first jaw and a second jaw, the first jaw having a first portion and a second portion that is opposite from the first portion; and a control configured to move at least one of the first jaw and the second jaw from a first configuration in which the first and the second jaws are spaced at a first distance, to a second configuration in which the first and the second jaws are spaced at a second distance that is less than the first distance.

Optionally, the second jaw comprises a first portion and a second portion, wherein the second portion of the second jaw is opposite from the first portion of the second jaw.

Optionally, the first jaw has a loop configuration, and the first and second portions of the first jaw are loop portions on opposite sides of the loop configuration.

Optionally, the first jaw has a C-shape.

Optionally, the control comprises a finger-loop and a thumb-loop.

Optionally, the medical tool further includes an elongated member disposed between the jaw assembly and the control.

Optionally, the first jaw is changeable from an extended configuration to a retracted configuration, and the first jaw is capable of being confined within a lumen of the elongated member when the first jaw is in the retracted configuration.

Optionally, the medical tool further includes an antenna configured to receive data transmitted by the endoscopic capsule.

Optionally, the first jaw is configured to provide a first magnetic field.

Optionally, the second jaw is configured to provide a second magnetic field.

Optionally, the first magnetic field is configured to assist in maintaining the endoscopic capsule at the certain position or within the range of positions relative to jaw assembly.

Optionally, the first jaw is configured to cooperate with the endoscopic capsule to deliver energy for treating a bleeding site at the bodily lumen.

Optionally, the first and second portions of the first jaw define a space therebetween for confining the endoscopic capsule.

Optionally, the jaw assembly has an opened configuration and a closed configuration, and wherein when the jaw assembly is in the closed configuration, the first jaw and the second jaw has a gap therebetween.

Optionally, the bodily lumen is in an intestine, and wherein the endoscopic capsule has a cross-sectional dimension D, and wherein the gap between the first jaw and the second jaw has a dimension that is anywhere from D1 to D2, where D1=2t, and D2<D+2t, t being a wall thickness of the intestine.

A medical system includes the medical tool described above, and the endoscopic capsule.

A medical system includes the medical tool described above, and a recorder for recording images transmitted from the endoscopic capsule.

A medical method that involves an intestine, includes: providing a jaw assembly having a first jaw and a second jaw, the first jaw having a first portion and a second portion, wherein the second portion is opposite from the first portion; and placing the first jaw and the second jaw on opposite sides of the intestine to grasp the intestine while an endoscopic capsule is inside the intestine, wherein the first portion of the first jaw is distal to a distal end of the endoscopic capsule, and the second portion of the first jaw is proximal to a proximal end of the endoscopic capsule.

Optionally, the method further includes moving the intestine relative to the endoscopic capsule while the endoscopic capsule images an internal wall of the intestine.

Optionally, the act of moving the intestine relative to the endoscopic capsule is accomplished by moving the intestine relative to the jaw assembly while the jaw assembly maintains the endoscopic capsule at a certain position or within a range of positions relative to the jaw assembly.

Optionally, the method further includes receiving image data transmitted from the endoscopic capsule.

Optionally, the act of receiving the image data is performed by an antenna at a device that includes the jaw assembly.

Optionally, the act of receiving the image data is performed by an antenna at a device that is separate from the jaw assembly.

Optionally, the method further includes marking or treating a bleeding site at the intestine after the bleeding site has been identified from the image data.

Optionally, the act of treating the bleeding site comprises delivering energy from the endoscopic capsule.

Optionally, the method further includes: inserting an elongated member through a first skin hole at a patient; and deploying the jaw assembly out of the elongated member from a retracted configuration to an extended configuration.

Optionally, the method further includes: inserting a grasper through a second skin hole at the patient; and using the grasper to move the intestine relative to the endoscopic capsule while the endoscopic capsule images an internal wall of the intestine.

Optionally, the method further includes: making a lengthwise incision at a skin of the patient; and opening the skin to expose the intestine.

Optionally, the jaw assembly has an opened configuration and a closed configuration, and wherein when the jaw assembly is in the closed configuration, the first jaw and the second jaw has a gap therebetween.

Optionally, the endoscopic capsule has a cross-sectional dimension D, and wherein the gap between the first jaw and the second jaw has a dimension that is anywhere from D1 to D2, where D1=2t, and D2<D+2t, t being a wall thickness of the intestine.

A method of manipulating an intestine includes: holding an exterior wall surface of the intestine; and moving the intestine relative to an endoscopic capsule located inside the intestine while the endoscopic capsule images an internal wall of the intestine.

Other and further aspects and features will be evident from reading the following detailed description.

### DESCRIPTION OF THE DRAWINGS

The drawings illustrate the design and utility of embodiments, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only exemplary embodiments and are not therefore to be considered limiting in the scope of the claims.

FIG. 1 shows a medical tool that includes a magnetic device in accordance with some embodiments.

FIG. 2 shows another medical tool in accordance with other embodiments.

FIG. 3 shows an endoscopic capsule in accordance with some embodiments.

FIGS. 4A and 4B shows other endoscopic capsules in accordance with other embodiments.

FIG. 5A shows a medical tool in a form of a laparoscopic device in accordance with some embodiments.

FIG. 5B shows a variation of the medical tool of FIG. 5A.

FIG. 5C shows a variation of the medical tool of FIG. 5A.

FIG. 5D shows a mechanism for adjusting an amount of gap between two jaws of a jaw assembly.

FIG. 6 shows an example of the medical tool of FIG. 5, particularly showing the medical tool mechanically maintaining the endoscopic capsule at a certain position.

FIG. 7 shows an example of the medical tool of FIG. 5, particularly showing the medical tool magnetically maintaining the endoscopic capsule at a certain position.

FIGS. 8A and 8B show how an intestine can be pulled over an endoscopic capsule.

FIG. 9 shows another example of the medical tool of FIG. 5, particularly showing the medical tool confining an endoscopic capsule.

FIGS. 10A-10C show variations of the medical tool of FIG. 9.

FIG. 11 shows a charging unit which may be separate from the capsule.

FIG. 12 shows an example where the capsule may be inductively charged during use within the patient body.

FIG. 13 shows one embodiment where the capsule may incorporate an insufflation system.

FIGS. 14A and 14B show another embodiment where the capsule may incorporate an expandable balloon around at least a portion of the capsule.

FIG. 15 shows a capsule with a window or opening through which laser light may be emitted.

### DETAILED DESCRIPTION OF THE INVENTION

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

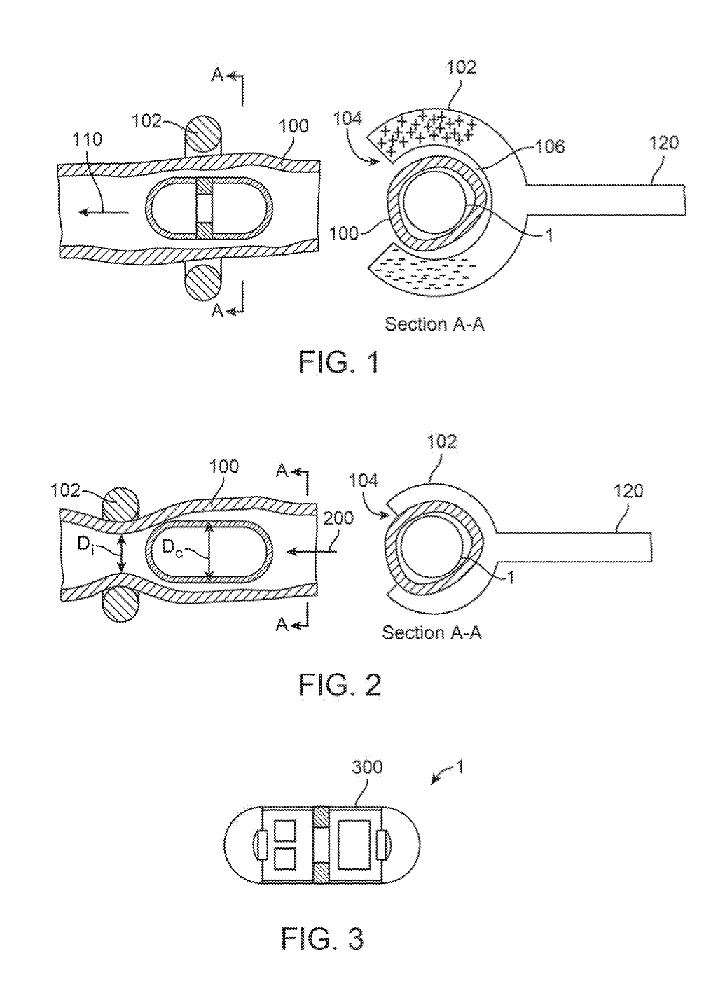

FIG. 1 shows a medical tool 2 that includes a magnetic device in accordance with some embodiments. The medical tool 2 is configured to maintain an endoscopic capsule 1 in an intestine 100 at a certain position relative to the medical tool 2. In particular, during one method of use, the GI tract of a patient is first cleared from residue, e.g. using a laxative. In another method of use, such as, during an emergency setting, the GI tract of the patient may not be cleared. Next, the endoscopic capsule 1 is swallowed by the patient, and enters into the GI tract. Alternatively, the endoscopic capsule 1 may be placed using gastroscope or surgically. The endoscopic capsule 1 then travels through the esophagus and the stomach, and enters into the small intestine. While inside the small intestine, the endoscopic capsule 1 performs capsule endoscopy (CE) and captures images of the interior of the small intestine. The image data is presented as real-time images for viewing by an operator so that condition (e.g., bleeding) in the small intestine may be identified. Optionally, the images may also be transmitted to a recorder outside the patient for recording. The capsule continues to travel through the patient's large intestine and the colon, and is retired during a normal bowel movement, typically within the next 2-3 days. In other cases, the capsule may be removed while a section of the intestine is resected.

As discussed, the medical tool 2 is configured to maintain the endoscopic capsule 1 at a certain position while the endoscopic capsule 1 is inside the patient. As shown in the figure, the medical tool 2 has a holder 102 configured to be placed around the intestine 100. The holder 102 has a C-configuration (an open-loop) with an opening 104 so that the intestine 100 can be placed in a space 106 defined by the holder 102 through the opening 104. It should be noted that the loop configuration for the holder 102 may be a completely or fully closed loop, or may be an opened loop (such as a loop with a slight gap). In the illustrated embodiments, the medical tool 2 is configured to maintain the endoscopic capsule 2 at a certain position using magnetic field. In particular, the holder 102 is configured to provide a magnetic field for interaction with the endoscopic capsule 2 so that the endoscopic capsule 2 stays at a certain position relative to the medical tool 2, and will not be allowed to travel downstream along the intestine 100 by the patient's body. For example, in some cases, the endoscopic capsule 1 may include a metallic component that allows the endoscopic capsule 1 to be pulled towards the medical tool 2 by the magnetic field provided by the medical tool 2. As another example, the endoscopic capsule 1 may include a magnetic component that allows the endoscopic capsule 1 to be pushed or pulled to a position by the magnetic field provided by the medical tool 2.

In some cases, the holder 102 of the medical tool 2 may include one or more permanent magnets for providing the magnetic field for interaction with the endoscopic capsule 1. In other cases, the holder 102 of the medical tool 2 may include a circuit coupled to an electrical source for providing electromagnetic field.

As shown in FIG. 1, the medical tool 2 also includes an elongated member 120 coupled to the holder 102. The elongated member 120 may be a handle for allowing a physician to move and position the holder 102. For example, in an open surgery, the physician may make a lengthwise incision on the skin of the patient, and may open up the skin to expose all or any portion of the intestine. Then the physician may position the holder 102 around the intestine 100 while the intestine 100 is directly visible to the physician. In other cases, the elongated member 120 may be a shaft sized for insertion through a skin opening at a patient. In such cases, the holder 102 may have a retracted configuration as the holder 102 is being inserted through the skin opening, and may extend or expand into an extended configuration after the holder 102 has passed through the skin inside the patient. This feature allows the medical tool 2 to be used as a laparoscopic tool to operate on the patient.

Referring to FIG. 1, once the configuration shown in the figure is achieved during a medical procedure, the position of the endoscopic capsule 1 is controlled by the medical tool 2, which prevents the endoscopic capsule 1 from travelling, such as in the downstream direction (represented by arrow 110), or in the upstream direction, along the length of the intestine 100. Next, an operator may then grasp a part of the intestine 100 and pull the intestine 100 relative to the endoscopic capsule 1. For example, in an open surgery, the operator may have a direct view of the intestine 100, and may physically pull the intestine 100 by hand. Alternatively, in a laparoscopic procedure, the operator may insert a grasper through a skin opening at the patient, and may use the grasper to grasp and pull the intestine 100 relative to the endoscopic capsule 1 while the medical tool 2 maintains the endoscopic capsule 1 at a certain position. It should be noted that the intestine can be moved relative to the capsule 1 in either or both directions (e.g., downstream and/or upstream). While the intestine 100 is being moved relative to the endoscopic capsule 1, the endoscopic capsule 1 captures images of the interior region of the intestine 100, and transmits the images to a screen outside the patient for viewing by an operator. The above technique is advantageous because it allows the operator to see real time images of the internal regions of the intestine 100. If a condition (e.g., bleeding) is identified from an image, the operator may readily stop the movement of the intestine 100 relative to the endoscopic capsule 1 to examine the region more carefully. Because the bleeding site may be identified in real time, and the location of the bleeding is known from such technique, an operator may immediately perform a treatment procedure to treat the bleeding condition. This technique is different and is advantageous over existing techniques in which the endoscopic capsule is allowed to freely move through the intestine by the patient's bodily function. In these existing techniques, even if a bleeding site is later identified from examining previously stored images, the location of the bleeding site along the intestine is unknown, and cannot be determined from the previously stored images.

FIG. 2 shows another medical tool 2 in accordance with other embodiments. The medical tool 2 is similar to that described with reference to FIG. 1, except that the medical tool 2 does not maintain the endoscopic capsule 1 in the intestine using magnetic field. Instead, the medical tool 2 is configured to maintain the endoscopic capsule 1 in the intestine mechanically. In particular, the holder 102 of the medical tool 2 may be positioned around the intestine that is downstream to the endoscopic capsule 1 (i.e., distal to the distal end of the endoscopic capsule 1). This way, as the patient's body attempts to move the endoscopic capsule 1 downstream (see arrow 200), the holder 102 will mechanically prevent the endoscopic capsule 1 from moving downstream. As shown in the figure, the holder 102 is configured to reduce a cross-sectional dimension of a lumen in the intestine to Di that is less than a cross sectional dimension Dc of the endoscopic capsule 1. This way, the holder 102 can mechanically prevent the endoscopic capsule 1 from moving downstream along the intestine.

FIG. 3 shows an endoscopic capsule 1 in accordance with some embodiments. The endoscopic capsule 1 includes a lighting system 3, a camera system 5, a magnet system 6, ends 7 that are transparent to light of appropriate wavelengths, a processing unit 4, a communication unit 8, and a power source 9. The lighting system 3 is configured to provide light to lit up region(s) in the intestine 100 that is being imaged. As shown in the figure, the lighting system 3 has two light sources pointing at opposite ends. This configuration allows both the region that is distal to the endoscopic capsule 1 and the region that is proximal to the endoscopic capsule 1 to be imaged. The camera system 5 is configured to capture images while the endoscopic capsule 1 is inside the intestine 100. In the illustrated embodiments, the camera system 5 includes a first camera and a second camera. The first camera is configured to capture images of region that is distal to the endoscopic capsule 1, and the second camera is configured to capture images of region that is proximal to the endoscopic capsule 1. In other embodiments, instead of having two light sources and two cameras, the endoscopic capsule 1 may include only one light source and only one camera for capturing images of a region inside the intestine 100 that is distal/proximal to the endoscopic capsule 1.

The processing unit 4 is configured to process images received from the camera system 5, and the communication unit 8 is configured to transmit images to an external recording device. In some cases, the communication unit 8 may include a transmitter for transmitting image data. In other cases, the communication unit 8 may include a transmitter and a receiver (e.g., a transceiver), in which cases, the communication unit 8 may receive data from another device (e.g., from the medical tool 2). The power source 9 is configured to provide power for operating the camera system 5, the lighting system 3, and the processing unit 4, and the communication unit 8. In some cases, the power source 9 may be a battery. In other cases, the power source 9 may be configured to generate power in response to energy transmitted by another device (e.g., the medical tool 2).

As shown in the figure, the endoscopic capsule 1 further includes a housing 300 for containing components of the endoscopic capsule 1. The housing 300 of the endoscopic capsule 1 may be made from a medical grade plastic, metal, or other types of materials.

The magnet system 6 is configured to provide one or more magnetic fields for interaction with the medical tool 2. In some embodiments, the magnet system 6 may include a magnetic component at the endoscopic capsule 1 configured to provide a magnetic field. The magnetic component may be implemented using one or more permanent magnets (e.g., one or more neodymium magnets), or an electromagnetic device. The magnetic component can be attached to an existing capsule (e.g., to an exterior surface or an interior surface of the housing 300). For example, the magnetic component may be a ring attached to the housing 300 of the endoscopic capsule 1. In other embodiments, a part of the housing 300 may be made from a magnetic material. Also, in some embodiments, the magnetic component may be made from a ferromagnetic material, such as iron.

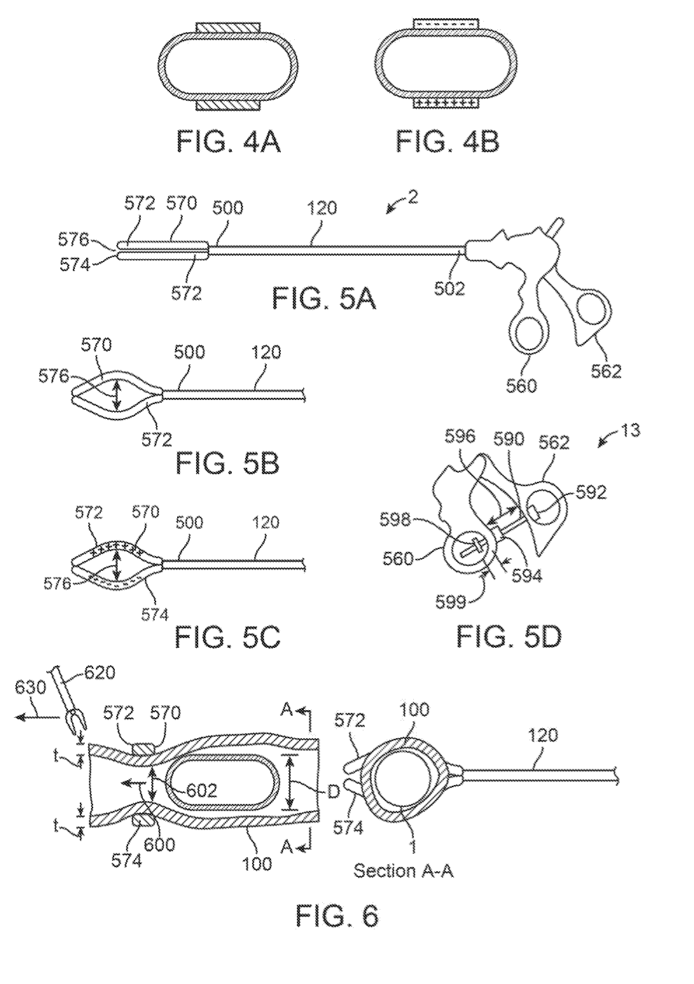

FIG. 4A shows another endoscopic capsule 1 in accordance with other embodiments. The endoscopic capsule 1 is similar to the endoscopic capsule 1 describe with reference to FIG. 3, except that the endoscopic capsule 1 does not have a magnetic system 6 that is inside the housing of the endoscopic capsule 1. Instead, the endoscopic capsule 1 has a metallic structure 11 that is coupled to the exterior surface of the endoscopic capsule 1. The metallic structure 11 allows the endoscopic capsule 1 to be attracted to a magnetic field, such as that provided by the medical tool 2. The metallic structure 11 may be secured to the exterior surface of the endoscopic capsule 1 by glue, adhesive, clip(s), pin(s), screw(s), or any of other types of securing devices. In some cases, the metallic structure 11 may have a ring configuration surrounding the housing 300 of the endoscopic capsule 1. In other embodiments, the metallic structure 11 itself may be a part of the housing 300 for the endoscopic capsule 1.

FIG. 4B shows another endoscopic capsule 1 in accordance with other embodiments. The endoscopic capsule 1 is similar to the endoscopic capsule 1 describe with reference to FIG. 3, except that the endoscopic capsule 1 does not have a magnetic system 6 that is inside the housing of the endoscopic capsule 1. Instead, the endoscopic capsule 1 has a magnetic structure 12 that is coupled to the exterior surface of the endoscopic capsule 1. The magnetic structure 12 allows the endoscopic capsule 1 to be reactive to a magnetic field, such as that provided by the medical tool 2. For example, the magnetic field from the medical tool 2 may push or pull the endoscopic capsule 1 to a certain desired position. The magnetic structure 12 may be secured to the exterior surface of the endoscopic capsule 1 by glue, adhesive, clip(s), pin(s), screw(s), or any of other types of securing devices.

As discussed, in some embodiments, the medical tool 2 may be in a form of a laparoscopic device. FIG. 5A shows the medical tool 2 in a form of a laparoscopic device in accordance with some embodiments. The medical tool 2 includes an elongated member 120 (e.g., a shaft) having a distal end 500 and a proximal end 502. The medical tool 2 also includes a jaw assembly 570 at the distal end 500 of the elongated member 120, and a control 13 at the proximal end 402 of the elongated member 120. The control 13 has a finger loop 560 and a thumb loop 562 for allowing a user to control the jaw assembly 570. The jaw assembly 570 has a first jaw 572 and a second jaw 574. The control 13 allows the user to open and close the jaw assembly 570 using scissor-like mechanism. For example, the control 13 may move only the first jaw 572 while the second jaw 574 is fixedly coupled to the shaft 120 to thereby open and/or close the jaw assembly 570. Alternatively, the control 13 may move only the second jaw 574 while the first jaw 572 is fixedly coupled to the shaft 120. In further embodiments, the control 13 may move both the first and second jaws 572, 574 relative to the shaft 120 to open and/or close the jaw assembly 570. In some embodiments, when the jaw assembly 570 is fully closed, the jaw assembly 570 has a gap 576 between the first jaw 572 and the second jaw 574. The gap 576 allows a section of an intestine to be slidably accommodated between the first and second jaws 572, 574 of the jaw assembly 570. For example, the gap 576 may have a value that is anywhere from D1 to D2, where D1=2t, D2<D+2t, t being a wall thickness of the intestine, and D being a cross sectional dimension of an endoscopic capsule to be controlled by the medical tool 2. In some cases, the gap 576 may be anywhere from 0.1 mm to 11 mm.

In some embodiments, the jaw assembly 570 may be fixedly secured to a distal end of the shaft 120. In such cases, the jaw assembly 570 may be made from a flexible and elastic material, which allows the jaw assembly 570 to be inserted through a trocar. Also, in any of the embodiments described herein, the jaw assembly 570 may be made from a flexible material that is sufficiently flexible to prevent tissue damage. In other embodiments, the jaw assembly 570 may be slidably coupled to the shaft 120. For example, the jaw assembly 570 may be retracted proximally into a lumen in the shaft 120, and may be advanced distally to deploy out of the lumen of the shaft 120. In such cases, the retraction and deployment of the jaw assembly 570 relative to the shaft 120 may be accomplished using a button at the control 13, which mechanically couples to the jaw assembly 570 and may be used to mechanically translate the jaw assembly 570 axially relative to the shaft 120. As shown in FIG. 5A, in some embodiments, when the jaw assembly 570 is deployed out of a lumen of the shaft 120, each of the first and second jaws 572, 574 may lie within a rectilinear plane. In particular, when viewed from a side, each of the first and second jaws 572, 574 has a rectilinear configuration 14 when the jaw assembly 570 is deployed outside the shaft 120.

In other embodiments, as shown in FIG. 5B, when the jaw assembly 570 is deployed outside the shaft 120, each of the first and second jaws 572, 574 may have a curvilinear configuration 15. In such cases, when the jaw assembly 570 is inside the lumen of the shaft 120, the jaw assembly 570 may have a confined and retracted configuration. When the jaw assembly 570 is deployed outside the lumen of the shaft 120, the jaw assembly 570 may assume an extended (or expanded) configuration. In one implementation, the jaw assembly 570 may be made from an elastic material, such as Nitinol, such that when the jaw assembly 570 is deployed outside the shaft 120, the jaw assembly 570 will automatically assume its extended configuration. In the illustrated embodiments, the jaw assembly 570 has a loop configuration with a gap 576 in the middle for accommodating a section of an intestine. As similarly discussed, the gap 576 may have a value that is anywhere from D1 to D2, where D1=2t, D2=D+2t, t being a wall thickness of the intestine, and D being a cross sectional dimension of an endoscopic capsule to be controlled by the medical tool 2.

FIG. 6 shows the medical tool 2 of FIG. 5B being used in a medical procedure to control a position of an endoscopic capsule 1. During use, an opening may be made through a patient's skin. Then the distal end of the shaft 120 may be inserted through the opening to reach a target site inside the patient, while the jaw assembly 570 is confined in a retracted configuration inside the shaft 120. When the distal end is manipulated so that it reaches the target site, the jaw assembly 570 is then deployed out of the distal end of the shaft 120. For example, a button at the control 13 may be operated to push the jaw assembly 570 out of the shaft 120. The jaw assembly 570 may be made from an elastic material, such as Nitinol, such that when the jaw assembly 570 is unconfined outside the shaft 120, the jaw assembly 570 automatically forms the extended configuration. The jaw assembly 570 is then operated using the finger loop 560 and the thumb loop 562 at the control 13 to open the jaw assembly 570, so that the first and second jaws 572, 574 may be placed on opposite sides of a section of an intestine. The finger loop 560 and thumb loop 562 may then be operated to close the jaw assembly 570 so that a part of the intestine is at least partially compressed, like that shown in FIG. 6. As shown in the figure, the endoscopic capsule 100 has a tendency to move downstream (represented by arrow 600) due to the bodily function of the intestine 100. Thus, in the illustrated embodiments, the part of the intestine 100 that is downstream from the endoscopic capsule 1 (i.e., distal to the distal end of the endoscopic capsule 1) is being compressed by the jaw assembly 570. In this configuration, the jaw assembly 570 in its closed configuration mechanically prevents the endoscopic capsule 1 from moving downstream because the gap 602 inside the intestine 100 is compressed to have a value that is less than the cross sectional dimension D of the endoscopic capsule 1.

After the configuration shown in FIG. 6 is achieved during the medical procedure, the intestine 100 may then be moved relative to the endoscopic capsule 1 while the endoscopic capsule 1 is maintained in the position relative to the medical tool 2. For example, as shown in FIG. 6, a grasper 620 may be inserted through a second opening at the patient's skin, and may be used to grasp a part of the intestine 100 that is distal to the part of the intestine being operated by the jaw assembly 570. The grasper 620 is then moved (e.g., pivoted about the location at the second skin opening) to pull the intestine 100 in the direction 630 while the jaw assembly 570 maintains the endoscopic capsule 1 at a certain position. As the intestine 100 is being pulled by the grasper 620, the intestine 100 slides relative to the jaw assembly 570, while the jaw assembly 570 prevents the endoscopic capsule 100 from moving downstream (see FIGS. 8A and 8B). Such technique allows the operator to manually move the intestine 100 relative to the endoscopic capsule 1. After the grasper 620 has moved the intestine 100 by a certain distance, the grasper 620 is then opened, and is then moved back closer to the jaw assembly 570 to grasp the intestine. The grasper 620 holding the intestine 100 is then again moved away from the jaw assembly 570 to pull the intestine 100 relative to the jaw assembly 570. This process is repeated until a desired length of the intestine 100 has been imaged by the endoscopic capsule 1. As the intestine 100 is being moved, the endoscopic capsule 1 captures images inside the intestine 100. In the illustrated embodiments, as the endoscopic capsule 1 views the interior of the intestine 100, the image data may be processed and may be displayed in a screen for viewing by the operator. The image data is presented in a form of real-time image, which allows the operator to determine a location of a bleeding inside the intestine.

In some cases, when the operator has identified a bleeding site in the intestine using the above technique, the operator may then mark the location with ink or a suture, and/or may immediately perform a treatment procedure to treat the bleeding site. Such is possible because the identification of the bleeding site is accomplished based on real time imaging. Thus, the location as viewed by the camera of the endoscopic capsule may be readily determined using the images captured by the camera. To treat a bleeding site, an operator may insert a treatment device laparoscopically through the patient's skin. The treatment device may then apply energy, for example, to stop the bleeding at the intestine. Additionally, or alternatively, the treatment device may apply a clip and/or suture to close off a bleeding wound at the intestine.

In some embodiments, the images may be transmitted wirelessly by the endoscopic capsule 1 to a receiving device that records the images. The receiving device may be a recorder with an antenna outside the patient. Alternatively, the receiving device may be a component at or coupled to the medical tool 2. For example, the jaw assembly 570 or the shaft 120 may include an antenna for receiving image data transmitted by the endoscopic capsule 1. In such cases, the medical tool 2 may include a recorder, or may be coupled to a recorder, for recording the image data. The recorded image allows the image data to be assessed later for review. For example, the recorded data may be analyzed by a nurse, a technician, a surgeon, a physician, a gastroenterologist, etc. In some cases, the image data may be in a form of a video stream. The viewing time may be reduced by increasing the playback speed. In some cases, the medical tool 2 may further include a processing unit for providing image processing. For example, the processing unit may include imaging processing module configured to perform automatic video summarization, automatic abnormality detection, pathology identification, topographic video segmentation, motion estimation, or any combination of the foregoing. Topographic video segmentation may be used to divide the video segments corresponding to the individual parts of the intestine. This technique can be enhanced using color information on different segments of the intestine. Motion estimation (visual odometry) may be used to reconstruct the camera motion in the intestine. This technique may be used to estimate the location and velocity of the camera.

In any of the embodiments described, the jaw assembly 570 may be configured to control a position of an endoscopic capsule mechanically without using any magnetic field. In other embodiments, the jaw assembly 570 may provide a magnetic field for magnetically controlling a position of an endoscopic capsule. For example, as shown in FIG. 5C, the first and second jaws 572, 574 may be configured to provide magnetic field(s) for interacting with an endoscopic capsule. In one implementation, the jaw assembly 570 may include permanent magnet(s) at the first and second jaws 572, 574. In another technique, the jaw assembly 570 may include circuit for providing electromagnetic field(s) through the first and second jaws 572, 574. Also, in further embodiments, the jaw assembly 570 itself may have portion(s) that is made from magnetic material.

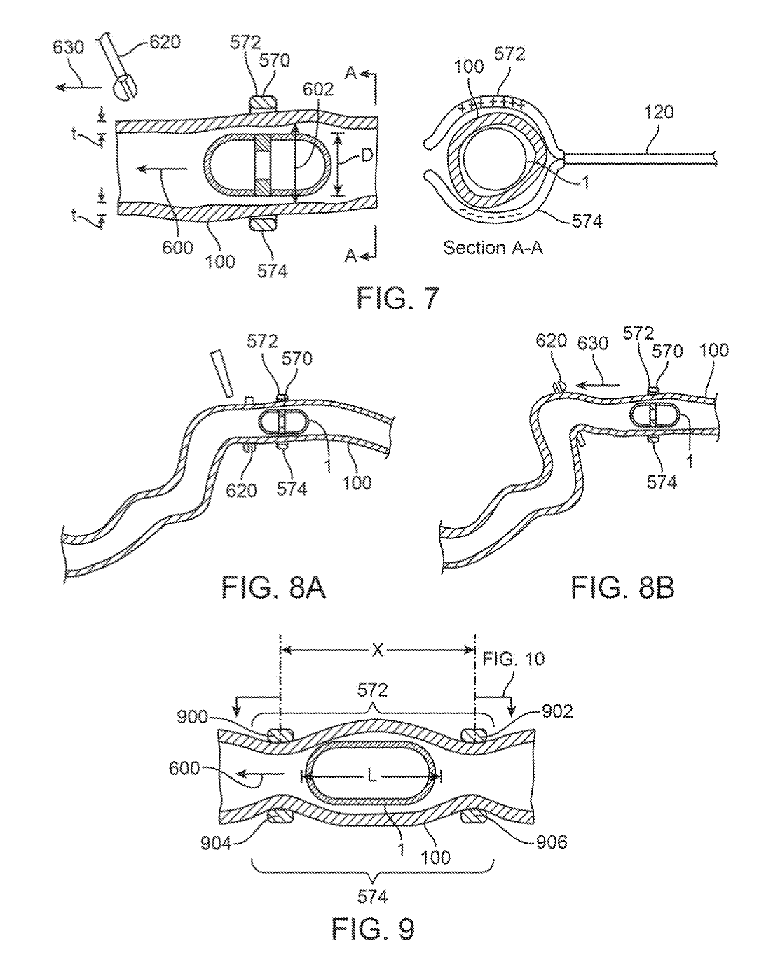

FIG. 7 shows the medical tool 2 of FIG. 5C being used in a medical procedure to control a position of an endoscopic capsule 1. During use, an opening may be made through a patient's skin. Then the distal end of the shaft 120 may be inserted through the opening to reach a target site inside the patient, while the jaw assembly 570 is confined in a retracted configuration inside the shaft 120. When the distal end is manipulated so that it reaches the target site, the jaw assembly 570 is then deployed out of the distal end of the shaft 120. For example, a button at the control 13 may be operated to push the jaw assembly 570 out of the shaft 120. The jaw assembly 570 may be made from an elastic material, such as Nitinol, such that when the jaw assembly 570 is unconfined outside the shaft 120, the jaw assembly 570 automatically forms the extended configuration. The jaw assembly 570 is then operated using the finger loop 560 and the thumb loop 562 at the control 13 to open the jaw assembly 570, so that the first and second jaws 572, 574 may be placed on opposite sides of a section of an intestine. The finger loop 560 and thumb loop 562 may then be operated to close the jaw assembly 570 so that a part of the intestine is at least partially compressed, like that shown in FIG. 7. As shown in the figure, the endoscopic capsule 100 has a tendency to move downstream (represented by arrow 600) due to the bodily function of the intestine 100. However, the endoscopic capsule 1 has a metal and/or magnetic field that interacts with magnetic field provided from the jaw assembly 570. In particular, the jaw assembly 570 in its closed configuration magnetically prevents the endoscopic capsule 1 from moving downstream because of its interaction with the metal and/or magnetic field at the endoscopic capsule 1. In the illustrated embodiments, the gap 602 inside the intestine 100 at the section of the intestine 100 being operated by the jaw assembly 570 may have a dimension that is larger than the cross sectional dimension D of the endoscopic capsule 1. In other embodiments, the gap 602 may be equal to the cross sectional dimension D, or may be less than the cross sectional dimension D, of the endoscopic capsule 1 (like that shown in FIG. 6).

After the configuration shown in FIG. 7 is achieved during the medical procedure, the intestine 100 may then be moved relative to the endoscopic capsule 1 while the endoscopic capsule 1 is maintained in the position relative to the medical tool 2. For example, as shown in FIG. 7, a grasper 620 may be inserted through a second opening at the patient's skin, and may be used to grasp a part of the intestine 100 that is distal to the part of the intestine being operated by the jaw assembly 570. The operation of the grasper 620 is similarly discussed with reference to FIG. 6, and therefore will not be repeated here.

In any of the embodiments described herein, the amount of the gap 576 provided at the jaw assembly 570 may be mechanically adjustable. For example, as shown in FIG. 5D, the control 13 may optionally further include a rod 590 fixedly secured to the thumb loop 562 by an anchor 592. The finger loop 560 may have a slot for allowing the rod 590 to extend therethrough, and the slot is slidable relative to the finger loop 560 during use. As shown in the figure, the control 13 of the medical tool 2 may also include a first nut 594 and a second nut 598 coupled to different respective positions along the length of the rod 590. The rod 590 may have screw threads for allowing the positions of the nuts 594, 598 to be selectively adjusted along the length of the rod 590. In particular, the first nut 594 is adjustable in position relative to the rod 590 so that a distance 596 between the finger loop 560 and the thumb loop 562 can be configured. The distance 596 prescribes the minimum distance the jaws 572, 574 will be apart from each other when the jaw assembly 570 is closed to prevent a traveling of the endoscopic capsule 1. On the other hand, the second nut 598 is for adjusting a distance 599 between the second nut 598 and the loop 560. The distance 599 prescribes the maximum distance the jaws 572, 574 will be apart from each other when the jaw assembly 570 is closed to prevent a traveling of the endoscopic capsule 1. In some embodiments, the nuts 594, 598 may configure the control 13 so that when the jaw assembly 570 is closed to operate with the endoscopic capsule 1, the gap 576 between the jaws 572, 574 will have a minimum distance of 2t (with t being a thickness of the wall of the intestine 100), and a maximum distance of D+2t (with D being a cross sectional dimension of the endoscopic capsule 1). In other embodiments, the maximum distance may be less than D+2t, such as D+t or D. During use, the second nut 598 may be operated (e.g., moved to the distal tip of the rod 590) to allow the jaw assembly 570 to be opened fully, so that the jaw assembly 570 may be placed around a section of the intestine 100. After the first and second jaws 572, 574 have been placed on opposite sides of the intestine 100, the jaw assembly 570 is then closed. At this stage, the second nut 598 may be moved proximally away from the tip of the rod 590 to thereby reduce the amount of gap 576 that can be achieved by moving the loops 560, 562 away from each other. The above feature is advantageous because it allows the medical tool 2 to slidably grasp the intestine 100 in a desired manner to prevent the endoscopic capsule 1 from moving downstream, without further input from the operator—i.e., the operator does not need to continuously apply compression force on the finger and thumb loops 560, 562 after the configuration of the jaw assembly 570 is set by the nuts 594, 598.

In the above embodiments, the medical tool 2 has been described as having a jaw assembly 570 configured to slidably grasp a section of the intestine 100 at a location that is distal to the distal end (i.e., downstream) of the endoscopic capsule 1. In other embodiments, the jaw assembly 570 may be configured to grasp a section of the intestine 100 simultaneously at two locations along the intestine 100. FIG. 9 shows another example of the medical tool 2 of FIG. 5, particularly showing the medical tool 2 confining an endoscopic capsule 1. In the illustrated embodiments, the first jaw 572 has a first portion 900 and a second portion 902 that is opposite from the first portion 900. The first portion 900 is for placement at a location that is distal to the distal end of the endoscopic capsule 1, and the second portion 900 is for placement at a location that is proximal to the proximal end of the endoscopic capsule 1. Similarly, the second jaw 574 has a first portion 904 and a second portion 906 that is opposite from the first portion 904. The first portion 904 is for placement at a location that is distal to the distal end of the endoscopic capsule 1, and the second portion 906 is for placement at a location that is proximal to the proximal end of the endoscopic capsule 1. As shown in the figure, the first portion 900 and the second portion 902 of the first jaw 572 are at a distance X away from each other, where X is equal to or greater than a length L of the endoscopic capsule 1. This allows the endoscopic capsule 1 to be confined between the first and second portions 900, 902 of the first jaw 572. Similarly, the first portion 904 and the second portion 906 of the second jaw 574 are at a distance X away from each other, where X is equal to or greater than a length L of the endoscopic capsule 1. This allows the endoscopic capsule 1 to be confined between the first and second portions 904, 906 of the first jaw 574. In some cases, the dimension X may be anywhere from 3 mm to 75 mm.

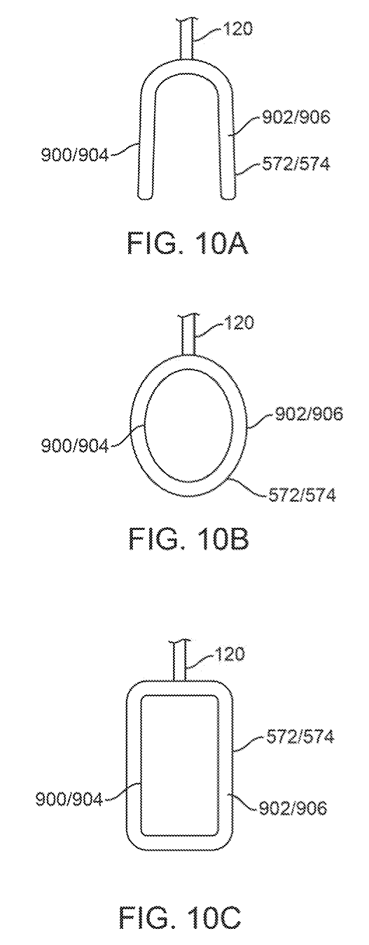

In some embodiments, each of the first and second jaws 572, 574 may have a C shape like that shown in FIG. 10A. In other embodiments, each of the first and second jaws 572, 574 may have a circular or elliptical shape like that shown in FIG. 10B. In such cases, the first portion 900 and second portion 902 of the first jaw 572 may be different segments along the length of a first circular/elliptical loop, and the first portion 904 and the second portion 906 of the second jaw 574 may be different segments along the length of a second circular/elliptical loop. In further embodiments, each of the first and second jaws 572, 574 may have a square or rectangular shape like that shown in FIG. 10C. In any of the above embodiments, the first and second jaws 572, 574 may have a retracted configuration when confined within a lumen of the shaft 120. The first and second jaws 572, 574 may be deployed out of the lumen of the shaft 120 to assume an extended configuration (like those shown in FIGS. 5A-5C). Alternatively, the jaw assembly 570 may be secured fixedly to the distal end of the shaft 120. In such cases, the jaw assembly 570 may be made from a flexible and elastic material, which allows the jaw assembly 570 to be inserted through a trocar. Also, in any of the embodiments described herein, the jaw assembly 570 may be made from a flexible material that is sufficiently flexible to prevent tissue damage.

It should be noted that the configurations described with reference to FIGS. 9 and 10 are advantageous because the medical tool 2 can confine the endoscopic capsule 1 to prevent the endoscopic capsule 1 from moving distally as well as proximally. This allows the relative positioning between the endoscopic capsule 1 and the surrounding intestine 100 to be achieved in a more precise manner.

In other embodiments, the jaw assembly 570 described with reference to FIGS. 9 and 10 may optionally further be configured to provide magnetic field, as similarly discussed.

In yet other embodiments, the endoscopic capsule 1 may incorporate an induction-based charging mechanism 1000 within the capsule 1 to provide power to the power storage unit (e.g., battery, capacitor) contained within. As shown in FIG. 11, a separate charging unit 1002 separate from the capsule 1 and located external to the patient's body may be positioned in proximity to the capsule 1 to provide an electromagnetic signal 1004 to the charging mechanism 1000. The capsule 1 may accordingly be charged prior to deployment in the patient or during use after the capsule 1 has already been ingested by the patient.

Alternatively, the capsule 1 may instead incorporate a set of antennas (e.g., monopole, dipole, yagi) for transferring energy into the capsule 1 power storage. In either case, the capsule 1 may be charged either prior to use in the patient or during use as well. Moreover, these charging embodiments may be used in combination with any of the other embodiments described herein.

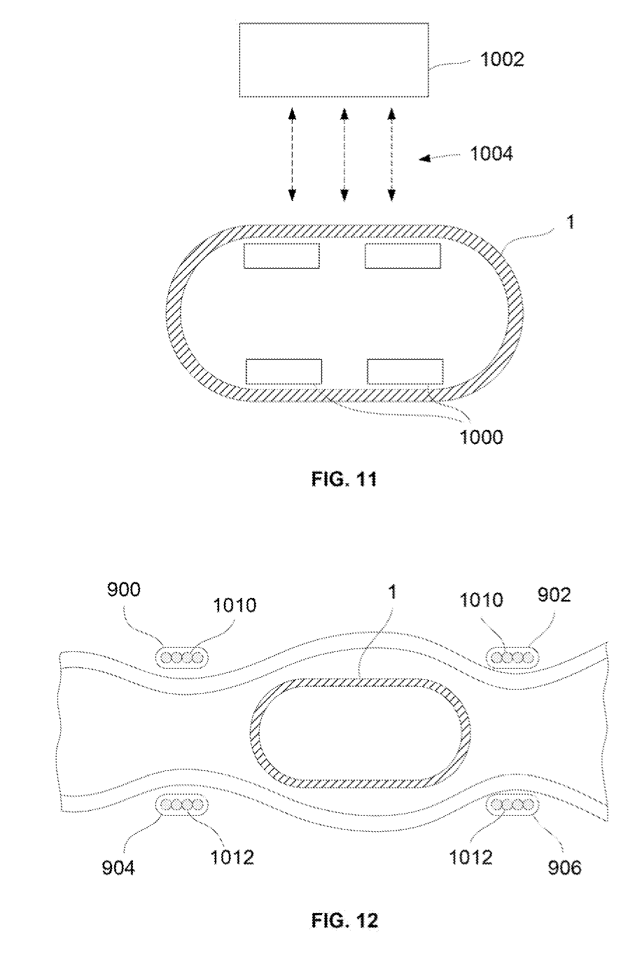

FIG. 12 shows an example where the capsule 1 may be inductively charged during use within the patient body by utilizing, e.g., the jaw assembly 570. The first portion 900 and second portion 902 of the first jaw are shown opposite respectively to the first portion 904 and second portion 906 of the second jaw. In this embodiment, the portions 900, 902 may integrate one or more loops 1010 and the apposed portions 904, 906 may likewise integrate one or more loops 1012. The loops 1010, 1012 may have a current passed through during use in the patient to provide power inductively to the capsule 1 retained within the patient body between the portions, as described above.

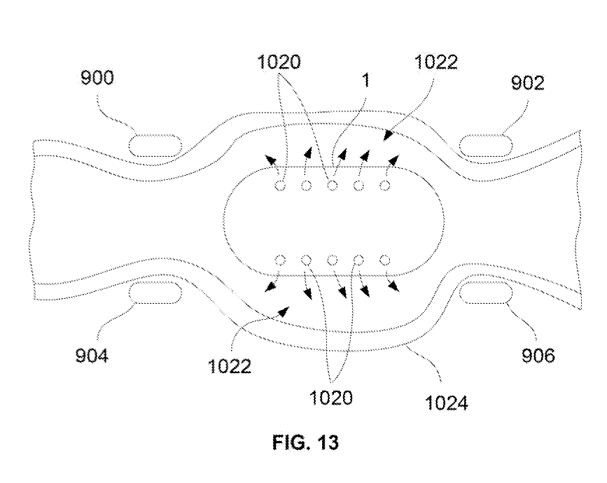

During use, there are occasions where visualizing the surrounding tissue or a particular region of tissue is made difficult due to anatomical features such creases or folds defined in the tissue. For these instances, it may be desirable or necessary to stretch a localized portion of tissue to enhance visualization of the area. With the capsule 1, certain features may be incorporated to allow for such visualization. For instance, FIG. 13 shows one embodiment where the capsule 1 may incorporate an insufflation system by storing a first compound and a second compound within for producing a chemical reaction when combined to produce a biocompatible gas. One example may include different compounds such as acetic acid and sodium bicarbonate to produce carbon dioxide gas. The compounds may be stored separately from one another within the capsule 1 but when actuated, the compounds may be mixed within the capsule 1 to produce the biocompatible gas 1022 which may exit the capsule through one or more exhaust ports 1020 defined along the capsule 1.

These compounds are provided as examples and any number of other compounds may be utilized to produce the biocompatible gas. Additionally, further examples of a capsule configured to produce a biocompatible gas is shown and described in further detail, e.g., U.S. Pub. 2014/0081169, which is incorporated herein by reference in its entirety and for any purpose. The exhausted gas 1022 may be retained locally around the capsule 1, e.g., by the jaw assembly which may prevent or inhibit the flow of the exhausted gas from escaping distally and/or proximally from the localized region, such that the surrounding tissue 1024 is expanded away from the walls of the capsule 1 to provide for an unobstructed view of the tissue walls by the capsule 1 camera. The gas may end production once the reaction has been completed and the local region may be vented simply by releasing the members of the jaw assembly so that the gas may be released. This released gas may be absorbed into the tissue or exit the patient naturally.

Alternatively, the capsule 1 may incorporate a pump which may also stretch the tissue as well. Such a pump may transfer the air and gas distal or proximal of the capsule 1 and the jaw members 900, 902 and 904, 906 and into the local region directly around the capsule itself.

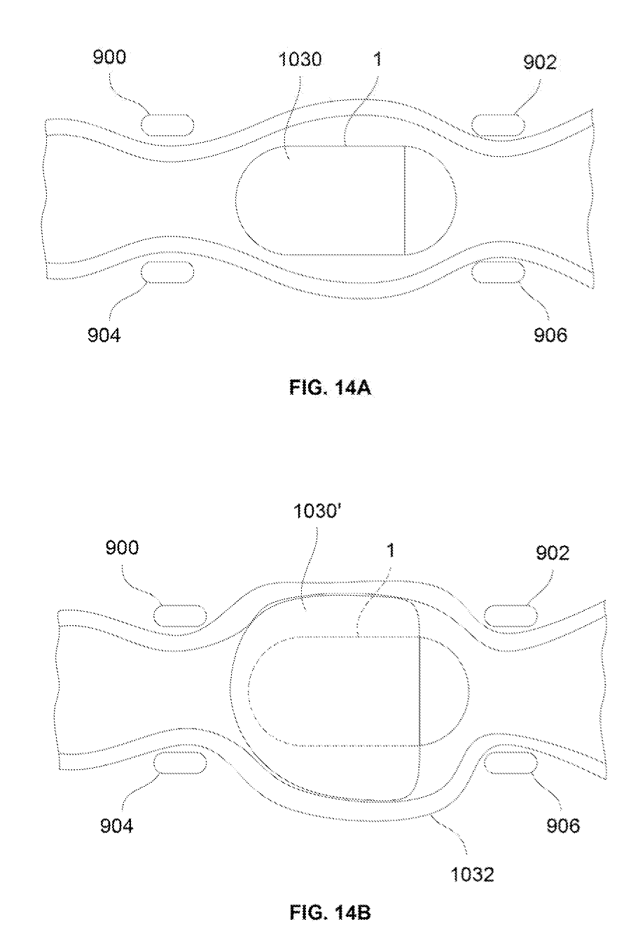

In yet another embodiment, the capsule may incorporate an expandable portion around an outer region of the capsule. FIGS. 14A and 14B show another embodiment where capsule 1 may incorporate an expandable balloon 1030 around at least a portion of the capsule 1. The balloon 1030 is shown in its deflated state in FIG. 14A where the balloon may be collapsed closely against the capsule 1. However, once inflated or expanded, the balloon 1030′ may be expanded away from the capsule 1 such that the balloon 1030′ contacts and expands against the interior tissue wall to distend it, as shown in FIG. 14B. The balloon 1030′ may be comprised of a distensible material and it may also be made to be translucent and/or transparent to allow for visualization of the underlying tissue through the expanded balloon 1030′, if needed. Further examples of inflatable balloons are shown and described in further detail in, e.g., U.S. Pub. 2011/0245611, which is incorporated herein by reference in its entirety and for any purpose.

The balloon 1030′ may be expanded by inflation through various methods such as a pump for infusing ambient air from around the capsule 1 and into the balloon 1030′. Alternatively, the balloon 1030′ may be used in combination with the creation of a biocompatible gas as described above.

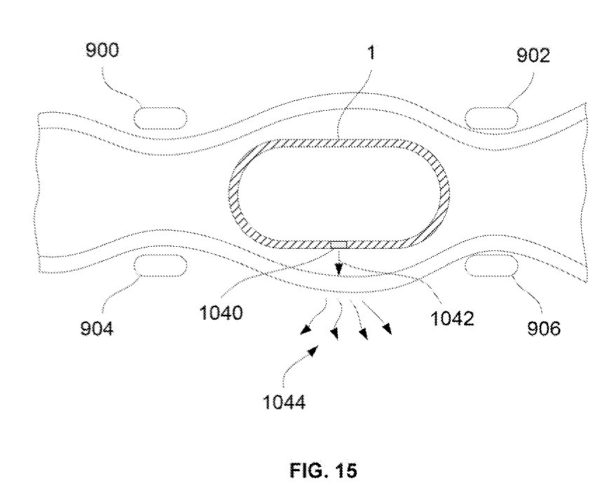

In yet another embodiment, the capsule 1 may further incorporate a light such as a laser which may be used to highlight a defect site from within the lumen so that the site is visible transluminally. Once the defect site is extraluminally visible, it may be marked or treated accordingly. An example is illustrated in the side view of FIG. 15 which shows a capsule 1 with a window or opening 1040 through which laser light 1042 may be emitted. In this example, the laser light 1042 may be emitted upon a defect site and the transluminally transmitted light 1044 may become visible extraluminally.

In yet other embodiments, the light emitted from the capsule may be color controlled so that the capsule 1 can be used to record images at different wavelengths. Transillumination at specific wavelengths can be used to visualize the anatomy and pathology of the lumen using, e.g., a laparoscopy imager.

In any of the embodiments described herein, the medical tool 2 may be a part of a medical system that includes the endoscopic capsule 1. Furthermore, any of these devices for improving visualization and diagnosis of the tissue wall may be used in any number of combinations with the features described herein and in U.S. patent application Ser. No. 15/160,410 filed May 20, 2016, which is incorporated herein by reference in its entirety and for any purpose.

Also, in any of the embodiments described herein, the medical tool 2 may be a part of a medical system that also includes a recording device for receiving images transmitted from the endoscopic capsule 1. In some cases, the recording device itself may have an antenna for wirelessly receive image data from the endoscopic capsule 1. In other cases, the medical tool 2 may include an antenna for wirelessly receive image data from the endoscopic capsule 1. In such cases, the medical tool 2 may include a recording device, or may be coupled to a recording device, that records the image data received by the antenna of the medical tool 2.

In addition, in any of the embodiments described herein the medical tool 2 may include an energy source for providing power for the endoscopic capsule 1 through the wall of the intestine. Such feature may be desirable as it may obviate the need to provide a battery for the endoscopic capsule 1.

Furthermore, in any of the embodiments described herein the medical tool 2 may be configured to cooperate with the endoscopic capsule 1 to provide energy to treat an interior wall of the intestine 100. For example, in some embodiments, the medical tool 2 may include a signal transmitter for transmitting a command signal to activate an energy source at the endoscopic capsule 1. The endoscopic capsule 1 may include a receiver for receiving the command signal, and the endoscopic capsule 1 with the energy source then provides energy to treat the intestine 100 in response to the command signal. As another example, the medical tool 2 may include a first electrode, and the endoscopic capsule 1 may include a second electrode. After a bleeding site has been identified in the intestine 100, the first and second electrodes may be energized to provide energy through the wall of the intestine 100 to thereby treat the intestine 100.

In the above embodiments, the systems and methods have been described with reference to detecting, locating, and treating a bleeding site in the intestine. However, it should be noted that the scope should not be limited to bleeding treatment. For example, the medical tools/systems and methods described herein may be used to detect, locate, and/or treat other conditions (e.g., tumor) in any tract (e.g., esophagus, small intestine, large intestine, colon, etc.) inside the patient.

Although particular embodiments have been shown and described, it will be understood that it is not intended to limit the claimed inventions to the preferred embodiments, and it will be obvious to those skilled in the art that various changes and modifications may be made without department from the spirit and scope of the claimed inventions. The specification and drawings are, accordingly, to be regarded in an illustrative rather than restrictive sense. The claimed inventions are intended to cover alternatives, modifications, and equivalents.

What is claimed is:

1. A tissue treatment system, comprising:
an endoscopic capsule which is sized for passage through an intestinal tract of a subject; and
a jaw assembly having at least a first jaw and a second jaw apposed to the first jaw, the jaw assembly presenting a surface for positioning upon an external surface of the intestinal tract,
wherein the jaw assembly further defines a region between the first and second jaws for maintaining the endoscopic capsule therebetween while the endoscopic capsule is within the intestinal tract, and
wherein the endoscopic capsule comprises an actuator for enhancing visualization within the intestinal tract.

2. The system of claim 1 wherein the first jaw has a loop configuration and the second jaw has a loop configuration apposed to the first jaw.

3. The system of claim 1 further comprising a recorder for recording images transmitted from the endoscopic capsule.

4. The system of claim 1 wherein the endoscopic capsule comprises a power supply which is configured for inductive charging.

5. The system of claim 4 wherein the first jaw and/or second jaw comprise one or more conductive loops for inductive charging of the power supply.

6. The system of claim 1 wherein the actuator comprises a first compound and a second compound which produce a biocompatible gas when combined for venting from the endoscopic capsule.

7. The system of claim 1 wherein the actuator comprises an expandable balloon disposed over at least a portion of the endoscopic capsule.

8. The system of claim 1 wherein the actuator comprises a laser which can be emitted from the endoscopic capsule.

9. A method for treating tissue within an intestinal tract, comprising:
introducing an endoscopic capsule into an intestinal tract of a subject;
positioning a jaw assembly having at least a first jaw and a second jaw around an external surface of the intestinal tract such that the endoscopic capsule is retained between the first jaw and second jaw;
maintaining a position of the endoscopic capsule relative to the subject via the jaw assembly while moving the intestinal tract; and
actuating the endoscopic capsule to enhance visualization within the intestinal tract.

10. The method of claim 9 wherein positioning a jaw assembly comprises positioning a loop configuration of the first jaw into apposition with a loop configuration of the second jaw about the external surface of the intestinal tract.

11. The method of claim 9 further comprising recording images transmitted from the endoscopic capsule.

12. The method of claim 9 further comprising charging a power supply within the endoscopic capsule by inductively charging the power supply through a wall of the intestinal tract.

13. The method of claim 12 where inductively charging comprises inductively charging via one or more conductive loops contained within the first jaw and/or second jaw.

14. The method of claim 1 wherein actuating comprises producing a biocompatible gas from within the endoscopic capsule and venting it into the intestinal tract such that at least a portion of the intestinal tract is insufflated.

15. The method of claim 1 wherein actuating comprises inflating a balloon positioned around at least a portion of the endoscopic capsule such that at least a portion of the intestinal tract is insufflated.

16. The method of claim 1 further comprising emitting a laser from the endoscopic capsule upon an internal surface of the intestinal tract.

17. The method of claim 16 further comprising extralumenally locating laser light transmitted through a tissue wall of the intestinal tract.
